(19) United States
(12) Patent Application Publication
(10) Pub. No.: US 2026/0002852 A1
(43) Pub. Date: Jan. 1, 2026

(54) SAMPLE FIXTURE FOR INDIRECT TENSILE TEST OF ROCK MASS

(71) Applicant: INSTITUTE OF GEOLOGY AND GEOPHYSICS, CAS, Beijing (CN)

(72) Inventors: Ning LIANG, Beijing (CN); Shengwen QI, Beijing (CN); Jianing CONG, Beijing (CN); Bowen ZHENG, Beijing (CN); Songfeng GUO, Beijing (CN); Yongchao LI, Beijing (CN); Lina MA, Beijing (CN); Wang ZHANG, Beijing (CN); Guoliang LI, Beijing (CN); Yanlong KONG, Beijing (CN); Yu ZOU, Beijing (CN); Xiaokun HOU, Beijing (CN); Zan WANG, Beijing (CN); Weiwei ZHU, Beijing (CN); Chao JIN, Beijing (CN); Wei LU, Beijing (CN); Guangming LUO, Beijing (CN); Xin YU, Beijing (CN); Xinyi GUO, Beijing (CN); Shuaihua SONG, Beijing (CN)

(21) Appl. No.: 18/912,964

(22) Filed: Oct. 11, 2024

(30) Foreign Application Priority Data
Jun. 27, 2024 (CN) 202410843796.0

(51) Int. Cl. G01N 3/08; G01N 3/06; G01N 33/24

(52) U.S. Cl. CPC G01N 3/08; G01N 3/06; G01N 33/24; G01N 2203/04

(57) ABSTRACT

Provided is a sample fixture for an indirect tensile test of rock mass, relating to the technical field of rock mass engineering geomechanics tests. The sample fixture includes an upper fixture, and a lower fixture located below the upper fixture. The upper fixture includes an upper fixing block, a ball head, and an upper knife. The lower fixture includes a lower fixing block, a telescopic positioning plate, and a lower knife. The ball head is in fit with a spherical groove on an upper surface of the upper fixing block to achieve the function of transferring pressure, thus ensuring loading strength acting on a sample. A loading force is transferred by using a cutting edge of the upper knife to make the pressure act on the sample intensively, thus ensuring point or linear loading required by the test.

### CROSS-REFERENCE TO RELATED APPLICATION

This patent application claims the benefit and priority of Chinese Patent Application No. 202410843796.0 filed with the China National Intellectual Property Administration on Jun. 27, 2024, the disclosure of which is incorporated by reference herein in its entirety as part of the present application.

### TECHNICAL FIELD

The present disclosure relates to the technical field of rock mass engineering geomechanics tests, and in particular to a sample fixture for an indirect tensile test of rock mass.

### BACKGROUND

Geotechnical engineering, such as mining engineering, geological disposal of nuclear wastes, water conservancy engineering and transportation engineering, is prone to rock tensile failure, which poses a serious security threat. Therefore, it is of a great significance to study the tensile strength of various rock masses for the safe development of the project. At present, the tensile strength of a rock is mainly studied by two types of indoor tests: a direct tensile test, and an indirect tensile test. Samples required for the two types of tests are different. The direct tensile test usually requires dog-bone-like or dumbbell-shaped samples which are difficult to manufacture, while the indirect tensile test usually uses cylindrical or cuboid samples which are easy to manufacture. At present, the direct tensile test is gradually replaced with the indirect tensile test using easily manufactured samples.

At present, in the indirect tensile test, a backing strip is usually placed at each of both ends of the sample, and then a relative linear load is applied to destroy the sample along the axial section, thus testing the tensile strength of the sample. However, the existing indirect tensile test has the following defects in the actual operation process: (1) In the loading process, because there may be some errors in the sample preparation process, it is difficult to ensure that the load acts strictly vertically on the sample, which will cause errors in the test results. (2) The current indirect tensile test instrument needs manual sample loading, it is difficult to ensure that the sample is centered in the indirect tensile instrument strictly, and thus the loading positions at both ends of the sample cannot be guaranteed. (3) The current indirect tensile test instrument has a single specification, which cannot be applied to samples with different shapes and sizes. For samples with different shapes and sizes, the instrument often needs to be modified, i.e., adding different types of backing strips, leading to difficult and time-consuming operation.

In conclusion, in the indirect tensile test, how to ensure that samples with different shapes and sizes can be centered in the indirect tensile instrument strictly and subjected to accurate vertical load is an urgent problem to be solved in the present disclosure.

### SUMMARY

A purpose of the present disclosure is to provide a sample fixture for an indirect tensile test of rock mass, which can ensure that samples with different shapes and sizes can be centered in an indirect tensile instrument strictly and subjected to an accurate vertical load, so as to solve the technical problems existing in the above existing indirect tensile test operation process, i.e., it is difficult to ensure that the samples are centered in the indirect tensile instrument strictly and the load acts on the samples strictly vertically, and the existing indirect tensile test instrument cannot be applied to samples with different shapes and sizes due to its single specification.

To achieve the purpose above, the present disclosure provides the following solutions: a sample fixture for an indirect tensile test of rock mass includes an upper fixture and a lower fixture. The upper fixture includes an upper fixing block, a ball head, and an upper knife, a spherical groove adapted for an outer arc surface of the ball head is formed on a top of the upper fixing block, and the ball head is embedded into the spherical groove; a bottom of the ball head is in contact and fits with the spherical groove, and a center of a contact surface between the ball head and the spherical groove is located at a central axis of the spherical groove; the upper knife is arranged below the upper fixing block, a cutting edge of the upper knife faces downwards, and the cutting edge of the upper knife is aligned with the central axis of the spherical groove; the ball head is configured to be connected to a loading power source to transfer a loading force exerted by the loading power source to the upper fixing block and the upper knife in sequence along the central axis of the spherical groove. The lower fixture is arranged below the upper fixture, and includes a lower fixing block, a telescopic positioning plate, and a lower knife; the lower knife is arranged on the lower fixing block, a cutting edge of the lower knife faces upwards, and the cutting edge of the lower knife is vertically aligned and parallel with the cutting edge of the upper knife; the telescopic positioning plate is located above the lower knife and the lower fixing block, and the telescopic positioning plate is connected to the lower fixing block by a telescopic adjusting assembly; the telescopic positioning plate is configured for placing a sample to be tested thereon, and a cutting-edge exposure hole, for the cutting edge of the lower knife to make contact with the sample to be tested, is formed in the telescopic positioning plate; a central line of the cutting-edge exposure hole in a length direction thereof is vertically aligned and parallel with the cutting edge of the lower knife; the telescopic adjusting assembly is configured for driving the telescopic positioning plate to move towards or away from the lower fixing block, so as to make the cutting edge of the lower knife in contact with or away from the sample to be tested on the telescopic positioning plate.

Preferably, when the sample to be tested is a cylindrical sample, the cutting-edge exposure hole also serves as a positioning structure for the cylindrical sample, the cylindrical sample is arranged parallel to the length direction of the cutting-edge exposure hole, and a lowermost part of the cylindrical sample is embedded into the cutting-edge exposure hole to limit both sides of the cylindrical sample by two long side edges of the cutting-edge exposure hole; and when the sample to be tested is a cuboid sample, positioning blocks are arranged on an upper surface of the telescopic positioning plate, the positioning blocks are distributed on an outer side of the cuboid sample to laterally limit the cuboid sample.

Preferably, the positioning blocks arranged on the upper surface of the telescopic positioning plate includes two positioning blocks, and the two positioning blocks are respectively located on two adjacent sides of the cuboid sample.

Preferably, the upper fixing block, the telescopic positioning plate and the lower fixing block are arranged in parallel, the upper fixing block is connected to the lower fixing block through a vertical guide mechanism, and the vertical guide mechanism is configured to vertically guide the upper fixing block when the upper fixing block moves up and down relative to the lower fixing block.

Preferably, the vertical guide mechanism includes multiple guide rods which are parallel to the central axis of the spherical groove, a bottom end of each of the multiple guide rods is connected to the lower fixing block, and a top end of each of the multiple guide rods movably penetrates through the upper fixing block to be in sliding fit with the upper fixing block; and the upper fixing block is connected to the lower fixing block by the multiple guide rods.

Preferably, upper fixing block side bumps are respectively arranged on two sides of the upper fixing block, and lower fixing block side bumps are respectively arranged on two sides of the lower fixing block; the lower fixing block side bumps are in one-to-one correspondence with the upper fixing block side bumps vertically; each of the multiple guide rods is connected between a corresponding one of the lower fixing block side bumps and a corresponding one of the upper fixing block side bumps, and a bearing is arranged in each of the upper fixing block side bumps, and each of the upper fixing block side bumps is in sliding fit with the top end of a corresponding one of the multiple guide rods by the bearing.

Preferably, the sample fixture for an indirect tensile test of rock mass further includes an axial strain sensor. The axial strain sensor is connected between the upper fixing block and the lower fixing block, and is configured to measure a displacement and deformation of the sample to be tested in a vertical direction in a loading process.

Preferably, sensor fixing seats are arranged on sides of both the upper fixing block and the lower fixing block, and the sensor fixing seats on the upper fixing block and the lower fixing block are in one-to-one correspondence vertically; and a top end and a bottom end of the axial strain sensor are respectively connected to the sensor fixing seats of the upper fixing block and of the lower fixing block.

Preferably, the telescopic adjusting assembly includes positioning holes, springs, and positioning shafts; the positioning holes are formed in the lower fixing block, the springs are embedded into the positioning holes, and bottom ends of the springs are coupled or connected to hole bottoms of the positioning holes; the positioning shafts are movably inserted into the positioning holes, top ends of the positioning shafts are connected to the telescopic positioning plate, and bottom ends of the positioning shafts are coupled or connected to top ends of the springs, or the bottom ends of the positioning shafts are inserted into the springs, limiting bumps are arranged on outer side walls of the positioning shafts, and the top ends of the springs are coupled or connected to the limiting bumps; the telescopic positioning plate is able to compress the springs under an action of gravity of the sample to be tested or the loading force exerted by the loading power source to enable the telescopic positioning plate to move towards the lower fixing block; and after the sample to be tested is taken down, the telescopic positioning plate is capable of being far away from the lower fixing block under a reset rebound effect of the springs.

Preferably, at least one of the cutting edge of the upper knife and the cutting edge of the lower knife is a round-headed cutting edge.

Compared with the prior art, the present disclosure achieves the following technical effects: a sample fixture for an indirect tensile test of rock mass provided by the present disclosure can ensure that a load loaded on the sample is perpendicular to the sample, and an action point is centered to ensure that the load acts on the sample in the center. The requirements of the indirect tensile test on the load are strictly satisfied, and the operation difficulty of the indirect tensile test is solved. The sample fixture is convenient in assembly, flexible to use, simple to operate, can be well compatible with samples with various shapes, and has strong applicability. The specific beneficial effects are as follows.

(1). The ball head is in fit with a spherical groove on an upper surface of the upper fixing block to achieve the function of transferring pressure, thus ensuring the loading strength acting on the sample.

(2). For a cylindrical sample, the centered placement of the sample is achieved by using the cutting-edge exposure hole, thus ensuring loading positions at both ends of the sample. For a cuboid sample, the centered placement of the sample can be achieved by using a sample groove enclosed by the positioning blocks on the telescopic positioning plate, thus ensuring the loading positions at both ends of the sample.

(3). The arrangement of the guide rod can ensure that the loading force is transferred vertically downward by the ball head, thus ensuring that the pressure can act on the center of the sample and at the loading position of the sample.

(4). The telescopic positioning plate cooperates with the spring, such that the sample can be stably and accurately placed on the telescopic positioning plate.

(5). The loading force is transferred by the cutting edge of the upper knife, such that the pressure can be concentrated on the sample to ensure point or linear loading required by the test.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a schematic diagram of an overall structure of a sample fixture according to Embodiment 1 of the present disclosure;

FIG. 2 is a front view of the sample fixture according to Embodiment 1 of the present disclosure;

FIG. 3 is a top view of the sample fixture according to Embodiment 1 of the present disclosure;

FIG. 4 is a longitudinal (parallel to a load loading direction) sectional view of the sample fixture according to Embodiment 1 of the present disclosure;

FIG. 5 is an enlarged schematic diagram of a structure at part A in FIG. 4;

FIG. 6 is an enlarged schematic diagram of a structure at part B in FIG. 4;

FIG. 7 is a schematic diagram of an overall structure of a sample fixture according to Embodiment 2 of the present disclosure;

FIG. 8 is a top view of the sample fixture according to Embodiment 2 of the present disclosure;

FIG. 9 is a front view of the sample fixture according to Embodiment 2 of the present disclosure;

FIG. 10 is a sectional view along a line C-C in FIG. 9;

FIG. 11 is a longitudinal (parallel to a load loading direction) sectional view of the sample fixture according to Embodiment 2 of the present disclosure; and

FIG. 12 is an enlarged schematic diagram of a structure at part D in FIG. 11.

In the drawings: 100 sample fixture for indirect tensile test of rock mass; 1 ball head; 2 bearing; 3 upper fixing block; 31 upper fixing block side bump; 4 upper knife; 5 guide rod; 6 axial strain sensor; 7 telescopic positioning plate; 71 cutting-edge exposure hole; 8 spring; 9 lower knife; 10 lower fixing block; 101 lower fixing block side bump; 11 sensor fixing seat; 12 positioning block; 13 cylindrical sample; 14 cuboid sample; 15 positioning hole; 16 positioning shaft.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

A purpose of the present disclosure is to provide a sample fixture for an indirect tensile test of rock mass, which can ensure that samples with different shapes and sizes can be centered in an indirect tensile instrument strictly and subjected to an accurate vertical load, so as to solve the technical problems existing in the existing indirect tensile test operation process, i.e., it is difficult to ensure that the samples are centered in the indirect tensile instrument strictly and the load acts on the samples strictly vertically, and the existing indirect tensile test instrument cannot be applied to samples with different shapes and sizes due to its single specification.

In order to make the purpose, features and advantages of the present disclosure more clearly, the present disclosure is further described in detail below with reference to drawings and embodiments.

### Embodiment 1

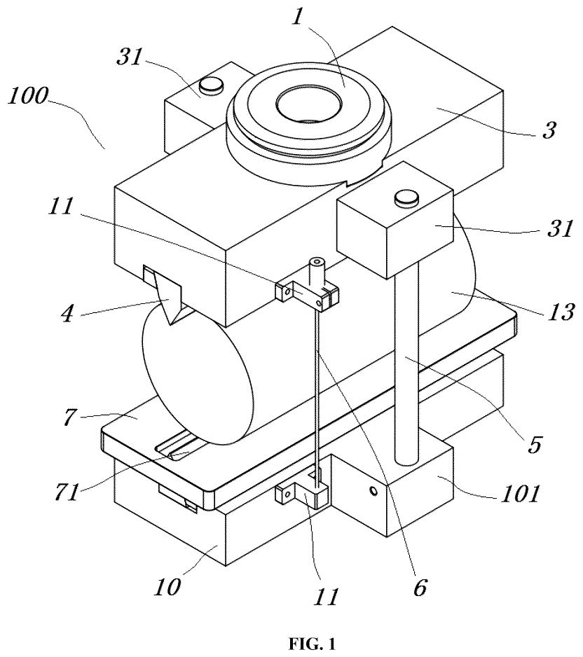

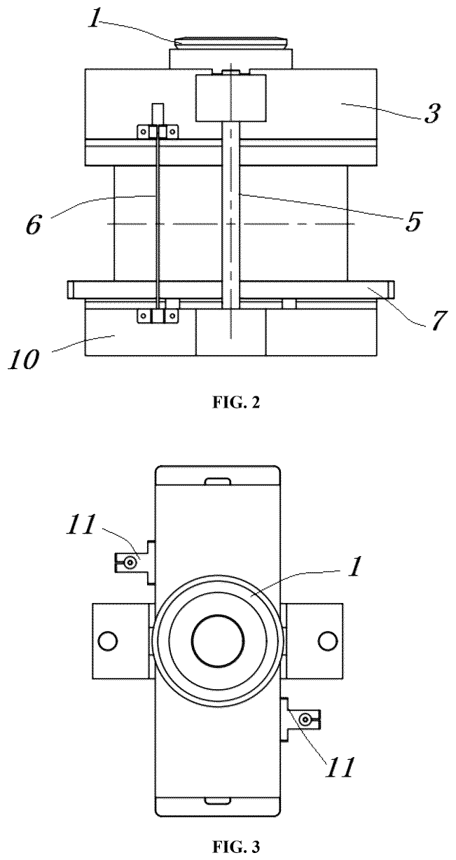

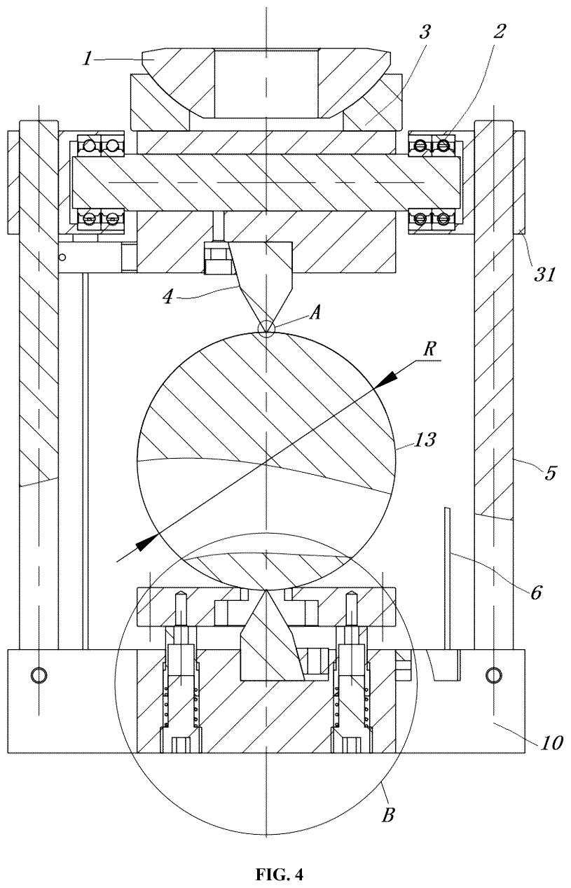

As shown in FIG. 1 to FIG. 6, a test fixture 100 for an indirect tensile test of rock mass provided by this embodiment includes an upper fixture, and a lower fixture located below the upper fixture. The upper fixture includes an upper fixing block 3, a ball head 1, and an upper knife 4. A spherical groove adapted for an outer arc surface of the ball head 1 is formed on the top of the upper fixing block 3, and the ball head 1 is embedded into the spherical groove. The bottom of the ball head 1 is in contact and fits with the spherical groove, and the center of a contact surface between the ball head 1 and the spherical groove is located at a central axis of the spherical groove. The upper knife 4 is arranged below the upper fixing block 3, a cutting edge of the upper knife 4 faces downwards, and the cutting edge of the upper knife 4 is aligned with the central axis of the spherical groove. The ball head 1 is configured to be connected to a loading power source (a telescopic mechanism such as a hydraulic machine), and the ball head 1 can transfer a loading force exerted by the loading power source to the upper fixing block 3 and the upper knife 4 in sequence along the central axis of the spherical groove. The lower fixture includes a lower fixing block 10, a telescopic positioning plate 7, and a lower knife 9. The lower knife 9 is arranged on the lower fixing block 10, a cutting edge of the lower knife 9 faces upwards, and the cutting edge of the lower knife 9 is aligned and parallel with the cutting edge of the upper knife 4. During actual test operation, a sample to be tested is clamped between the cutting edge of the lower knife 9 and the cutting edge of the upper knife 4, and a length direction (an axial direction of the sample) of the sample to be tested is parallel to extending directions of the cutting edge of the lower knife 9 and the cutting edge of the upper knife 4. The telescopic positioning plate 7 is located above the lower knife 9 and the lower fixing block 10, and the telescopic positioning plate 7 is connected to the lower fixing block 10 through the telescopic adjusting assembly. The telescopic positioning plate 7 is configured for placing the sample to be tested thereon, and a cutting-edge exposure hole 71, for the cutting edge of the lower knife 9 to make contact with the sample to be tested, is formed in the telescopic positioning plate 7. The cutting-edge exposure hole 71 is a strip-shaped hole, and a length direction of the cutting-edge exposure hole 71 is parallel to extending directions of the cutting edge of the lower knife 9 and the cutting edge of the upper knife 4, and a central line of the cutting-edge exposure hole 71 in a length direction thereof is vertically aligned and parallel with the cutting edge of the lower knife 9. The telescopic adjusting assembly can make the telescopic positioning plate 7 and the lower fixing block 10 to be close to each other or far away from each other, thus making the cutting edge of the lower knife 9 reach the cutting-edge exposure hole 71 to make contact with the sample to be tested, or making the cutting edge of the lower knife 9 away from the cutting-edge exposure hole 71, so as to be away from the sample to be tested on the telescopic positioning plate 7. According to the above sample fixture, the ball head 1 is in contact with the spherical groove on the upper fixing block 3, which can ensure that the ball head 1 moves in a vertical direction under the action of loading, and ensure that a load transferred from the ball head 1 to the upper fixing block 3 after receiving the load is always perpendicular to the upper fixing block 3 in any test. The upper fixing block 3 constrains the position of the ball head 1 through the spherical groove and fixes the upper knife 4, thus ensuring that the ball head can be centrally pressed against the cutting edge of the upper knife 4. The sample is clamped by the upper knife 4 and the lower knife 9, thus achieving that a load applied to upper side and lower side of the sample is a linear load. Moreover, the cutting edges of the upper and lower knives are suitable for samples with various sizes and shapes without frequent replacement. Therefore, in general, welding technology is preferred to permanently connect the lower fixing block 10 and the lower knife 9, as well as the upper fixing block 3 and the upper knife 4, which can prevent a situation that the position deviation of the upper and lower knives in the test process affects the test result while simplifying the fixture structure and assembly steps.

In this embodiment, taking the sample to be tested as a cylindrical sample 13 as an example, during the indirect tensile test, the cylindrical sample 13 is arranged above and parallel to the cutting-edge exposure hole 71, and a diameter of the cylindrical sample 13 is much greater than a width of the cutting-edge exposure hole 71, and an axial length of the cylindrical sample 13 is generally shorter than a length of the cutting-edge exposure hole 71. Based on the structural characteristics of the cylindrical surface of the cylindrical sample 13 and the self-weight of the cylindrical sample 13, when the cylindrical sample 13 is placed on the telescopic positioning plate 7, the bottom of the cylindrical sample 13 is embedded into the cutting-edge exposure hole 71. At this time, the central axis of the cylindrical sample 13 is perpendicular to and intersects with the central axis of the spherical groove, and the cutting-edge exposure hole 71 also serves as a positioning structure for the cylindrical sample 13. The cutting-edge exposure hole 71 can limit both sides of the cylindrical sample 13 through two long side edges of the cutting-edge exposure hole 71 to ensure the stability of the cylindrical sample.

In this embodiment, the upper fixing block 3, the telescopic positioning plate 7 and the lower fixing block 10 are preferably arranged in parallel, and the upper fixing block 3 is connected to the lower fixing block 10 through a vertical guide mechanism. The vertical guide mechanism is configured to vertically guide the upper fixing block 3 when the upper fixing block 3 moves up and down relative to the lower fixing block 10 under the driving of the loading power source. Specifically, the vertical guide mechanism includes guide rods 5 which are parallel to the central axis of the spherical groove, a bottom end of each guide rod 5 is connected to the lower fixing block 10, and a top end of the guide rod 5 movably penetrates through the upper fixing block 3 to be in sliding fit with the upper fixing block 3. The upper fixing block 3 and the lower fixing block 10 are connected through multiple guide rods 5. The multiple guide rods 5 are preferably distributed on different sides of the upper fixing block 3 and the lower fixing block 10, as shown in FIG. 1. The upper fixing block 3 is connected to the lower fixing block 10 through the two guide rods 5, and the two guide rods 5 are respectively located on two sides of the upper fixing block 3 and the lower fixing block 10. The guide rod 5 can restrain a movement track of the ball head 1 under the action of the load, thus ensuring that the ball head 1 keeps moving vertically downward under the load, and ensuring that the load acts on the sample in the loading process.

As a further preferred solution, in this embodiment, upper fixing block side bumps 31 are respectively arranged on two sides of the upper fixing block 3, and lower fixing block side bumps 101 are respectively arranged on two sides of the lower fixing block 10. The lower fixing block side bumps 101 are in one-to-one correspondence with the upper fixing block side bumps 31 vertically. Each guide rod 5 is connected between a corresponding one of the lower fixing block side bumps 101 and a corresponding one of the upper fixing block side bumps (31). For example, two guide rods 5 are provided, each of the two sides of the upper fixing block 3 is provided with an upper fixing block side bump 31, correspondingly, each of the two sides of the lower fixing block 10 is provided with a lower fixing block side bump 101. The two guide rods 5 are connected to two pairs of lower fixing block side bumps 101 and upper fixing block side bumps 31 on the two sides of the upper fixing block and the lower fixing block. A length of each guide rod 5 is determined by a diameter size R of the cylindrical sample 13 required by the test. Therefore, the guide rod 5 needs to be frequently replaced in different tests. Therefore, it is preferable that the bottom end of the guide rod 5 is not permanently connected to the lower fixing block side bump 101 to facilitate subsequent replacement.

Further, a bearing 2 is arranged in each upper fixing block side bump 31, and each upper fixing block side bump 31 is in sliding fit with the top end of the guide rod 5 through the bearing 2. The bearing 2 is preferably a linear slide bearing, which can reduce a friction force between the upper fixing block side bump 31 and the guide rod 5, and transfer the load to a test sample with less loss.

In this embodiment, an axial strain sensor 6 is further provided. The axial strain sensor 6 is connected between the upper fixing block 3 and the lower fixing block 10, and is configured to measure a displacement and deformation of the sample to be tested in a vertical direction (i.e., A direction perpendicular to an axial direction of the sample to be tested, or a direction parallel to the central axis of the spherical groove) in the loading process. Further, in this embodiment, a side edge of each of the upper fixing block 3 and the lower fixing block 10 is provided with a sensor fixing seat 11, and the sensor fixing seat 11 on the upper fixing block 3 and the sensor fixing seat 11 on the lower fixing block 10 are in one-to-one correspondence vertically, as shown in FIG. 1. A top end and a bottom end of the axial strain sensor 6 are respectively connected to the sensor fixing seat 11 of the upper fixing block 3 and the sensor fixing seat 11 of the lower fixing block 10. Specifically, a side wall of each of the upper fixing block 3 and the lower fixing block 10 is reserved with threaded holes, the sensor fixing seats 11 are fixed to the threaded holes in the upper fixing block 3 and the lower fixing block 10 by corresponding nuts, thus installing the axial strain sensor 6 for measuring axial strain in the subsequent test process.

In this embodiment, the telescopic adjusting assembly includes positioning holes 15, springs 8, and positioning shafts 16. The positioning holes 15 are formed in an upper surface of the lower fixing block 10, the springs 8 are embedded into the positioning holes 15, and bottom ends of the springs 8 are coupled or connected to hole bottoms of the positioning holes 15. The positioning shafts 16 are movably inserted into the positioning holes 15 and capable of sliding relative to the positioning holes 15. Top ends of the positioning shafts 16 are connected to the telescopic positioning plate 7, bottom ends of the positioning shafts 16 are located to the top ends of the springs 8, and bottom end faces of the positioning shafts 16 are coupled or connected to the top ends of the springs 8. Or the bottom ends of the positioning shafts 16 are inserted into the springs 8. Outer side walls of the positioning shafts 16 are provided with limiting bumps, and the top ends of the springs 8 are coupled or connected to the limiting bumps. The telescopic positioning plate 7 can compress the springs 8 under the action of the gravity of the sample to be tested or the loading force exerted by the loading power source to enable the telescopic positioning plate 7 to move towards the lower fixing block 10, so that the cutting edge of the lower knife 9 reaches the cutting-edge exposure hole 71 to make contact with the sample to be tested. In general, the telescopic positioning plate 7 can be pressed down under the own gravity of the sample to be tested, and then the spring 8 is compressed to enable the cutting edge of the lower knife 9 below the telescopic positioning plate 7 to be in contact with the sample to be tested. After the sample to be tested is taken down from the telescopic positioning plate 7, the telescopic positioning plate 7 can rise under the reset rebound effect of the springs 8 and move away from the lower fixing block 10, such that the cutting edge of the lower knife 9 will be away from the cutting-edge exposure hole 71 and then away from the sample to be tested on the telescopic positioning plate 7. The telescopic adjusting assembly cooperates with the telescopic positioning plate 7, such that the sample can be accurately placed when the fixture does not clamp the sample.

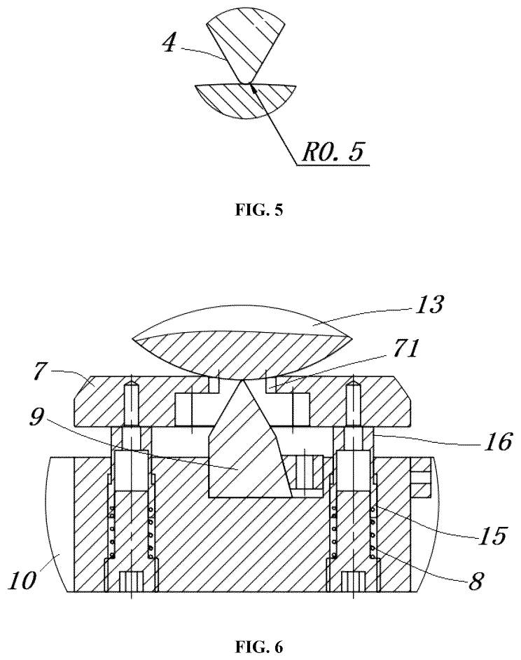

In this embodiment, preferably, at least one of the cutting edges of both the upper knife 4 and the lower knife 9 is a round-headed cutting edge. FIG. 5 is a structural diagram of a round-headed cutting edge of the upper knife 4, the round-headed cutting edge is preferably a round head with a radius of 0.5 mm, which can form a linear load at a position in contact with the sample, thus achieving a load required by the indirect tensile test.

In this embodiment, both the upper fixture and the lower fixture are preferably axisymmetric structures, the axes of the upper fixture and the lower fixture coincide with each other and coincide with a center line of the sample in each test. The entire fixture can be repeatedly mounted and dismounted, and meanwhile, the fixture is small to facilitate the movement in the later handling process.

Taking the indirect tensile test of rock mass with the cylindrical sample 13 as an example, the assembly process and use principle of the sample fixture 100 for the indirect tensile test of the rock mass are described in detail below.

Prior to test, the test fixture 100 for an indirect tensile test of rock mass and the cylindrical sample 13 need to be assembled, the lower fixture is installed at first, and the lower fixing block 10 is placed on a horizontal platform, a sensor fixing seat 11 is installed to threaded holes of a side wall of the lower fixing block 10, and the springs 8 are placed in the positioning holes 15 at the top of the lower fixing block 10. The positioning shafts 16 at the bottom of the telescopic positioning plate 7 extend into the positioning holes 15 and are coupled or connected to the springs 8. At this time, the axis of the telescopic positioning plate 7 coincides with the axis of the lower fixing block 10, and the cutting-edge exposure hole 71 is arranged at the center of the telescopic positioning plate 7.

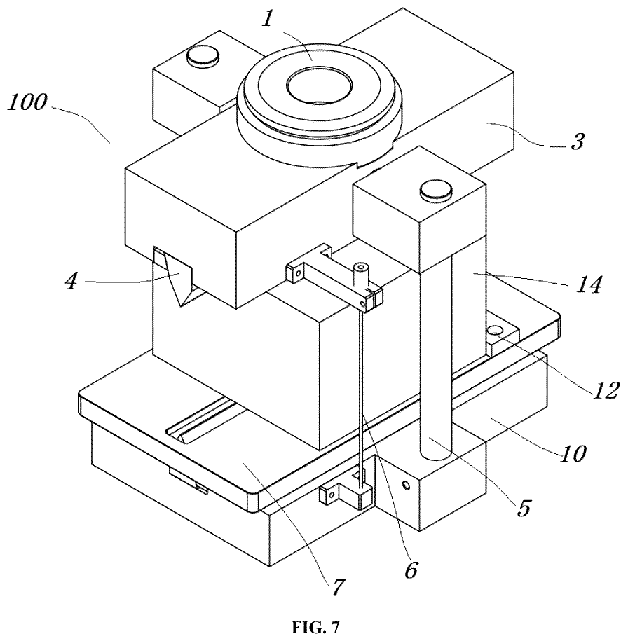

Then, according to the size of the cylindrical sample 13 required by the test, the corresponding guide rods 5 are selected to be installed between the lower fixing block 10 and the upper fixing block 3, and it is necessary to ensure that the guide rods 5 are stable and do not move during mounting. The guide rod 5 is mounted, and the prepared cylindrical sample 13 is placed on the upper portion of the telescopic positioning plate 7. The cylindrical sample 13 is firstly in contact with two sides of the cutting-edge exposure hole 71, and the axis of the sample coincides with the axis of the lower fixing block 10 through the self-weight of the cylindrical sample 13. The cylindrical sample 13 drives the telescopic positioning plate 7 vertically downwards under the self-weight of the cylindrical sample 13 until the bottom of the cylindrical sample 13 is abutted against the cutting edge of the lower knife 9 as shown in FIG. 6, and the telescopic positioning plate 7 no longer moves.

Then, the upper fixture is mounted, the upper fixing block 3 is mounted on the guide rods 5, thus ensuring that the upper fixing block 3 can move freely along the guide rods 5 after the upper fixing block 3 is installed. Prior to test, it should be ensured that the upper knife 4 of the upper fixing block 3 is far away from the sample, or slowly approaches the cylindrical sample 13, thus preventing the cylindrical sample 13 from being damaged before the test starts.

If an axial strain needs to be measured, the axial strain sensor 6 needs to be mounted between the sensor fixing seats 11 of the upper fixing block 3 and of the lower fixing block 10.

The mounted fixture and the cylindrical sample 13 are placed on an indirect tensile tester, the ball head 1 is placed in the spherical groove on the upper portion of the upper fixing block 3, the indirect tensile test can be carried out.

The sample fixture 100 for an indirect tensile test of rock mass provided by the present disclosure solves the operation difficulty of the indirect tensile test, which is convenient in assembly, flexible in use, simple in operation, can be well compatible with samples with various shapes, and has strong applicability. The present disclosure achieves the beneficial effects as follows.

The ball head 1 is in fit with a spherical groove on an upper surface of the upper fixing block 3 to achieve the function of transferring pressure, thus ensuring loading strength acting on the sample.

For a cylindrical sample, the centered placement of the sample is achieved by using the cutting-edge exposure hole, thus ensuring loading positions at both ends of the sample. For a cuboid sample, the centered placement of the sample can be achieved by using a sample groove enclosed by the positioning blocks on the telescopic positioning plate 7, thus ensuring the loading positions at both ends of the sample.

The arrangement of the guide rod can ensure that the loading force is transferred vertically downward by the ball head, thus ensuring that the pressure can act on the center of the sample and at the loading position of the sample.

The telescopic positioning plate cooperates with the spring, such that the sample can be stably and accurately placed on the telescopic positioning plate.

The loading force is transferred by the cutting edge of the upper knife, such that the pressure can be concentrated on the sample to ensure point or linear loading required by the test.

According to this fixture, the sample can be mounted before the indirect tensile test, thus reducing the preparation prior to the test in a test room.

According to this fixture, it is can be ensured that the load loaded on the sample is perpendicular to the sample, and the action point is centered, so as to ensure that the load acts on the center of the sample, and the requirements of the indirect tensile test on the load are strictly satisfied.

### Embodiment 2

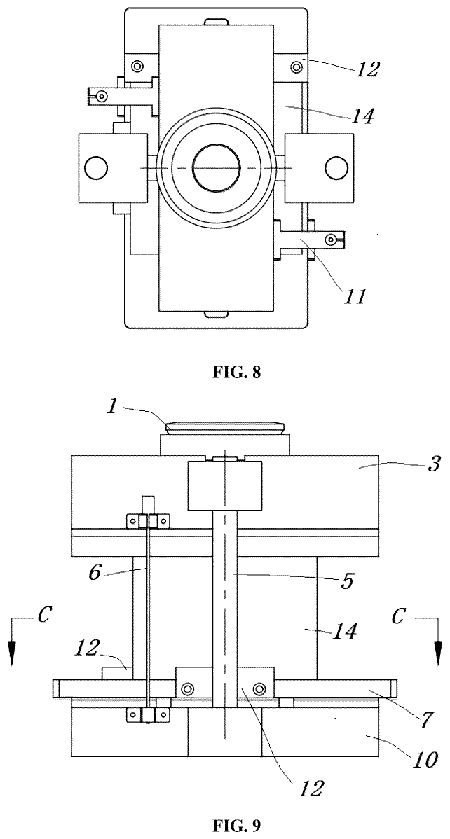

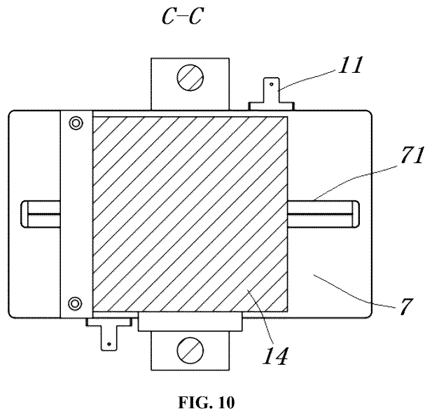

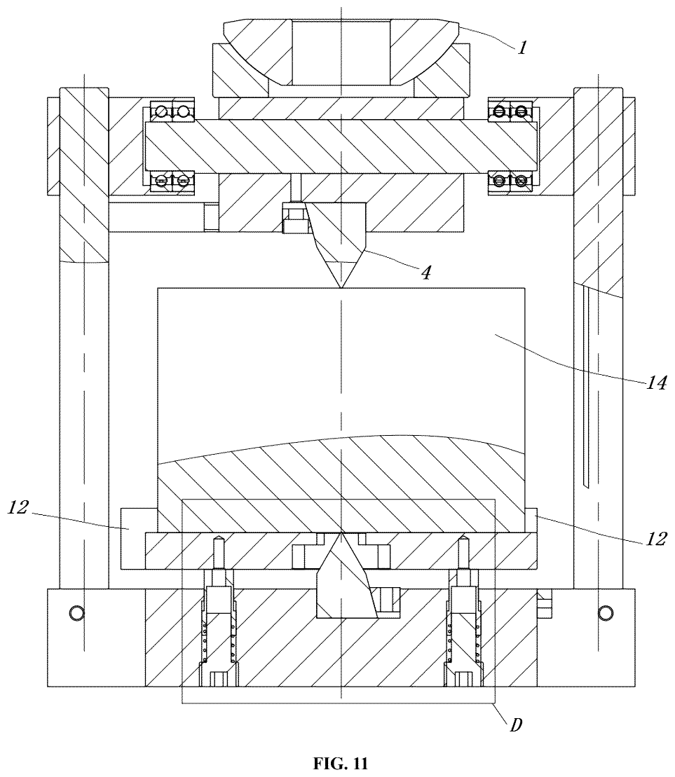

As shown in FIG. 7 to FIG. 12, a test fixture 100 for an indirect tensile test of rock mass is provided in this embodiment, the difference between this embodiment and Embodiment 1 is that the test fixture of Embodiment 1 is suitable for a cylindrical sample 13, while the test fixture in this embodiment is mainly suitable for a cuboid sample 14. Specifically, this embodiment is for the cuboid sample 14. Positioning blocks 12 are further arranged on the upper surface of the telescopic positioning plate 7, and the positioning blocks 12 are distributed on outer sides of the cuboid sample 14, thus laterally limiting the cuboid sample 14. At this time, the telescopic positioning plate 7 is inconsistent with the telescopic positioning plate 7 suitable for the cylindrical sample in Embodiment 1, the cuboid sample is difficult to be centrally placed, such that the positioning blocks 12 can be enclosed on the telescopic positioning plate 7 to form a sample groove for indicating the mounting of the cuboid sample, and the telescopic positioning plates 7 with sample grooves of different sizes can be replaced according to cuboid samples 14 of different sizes.

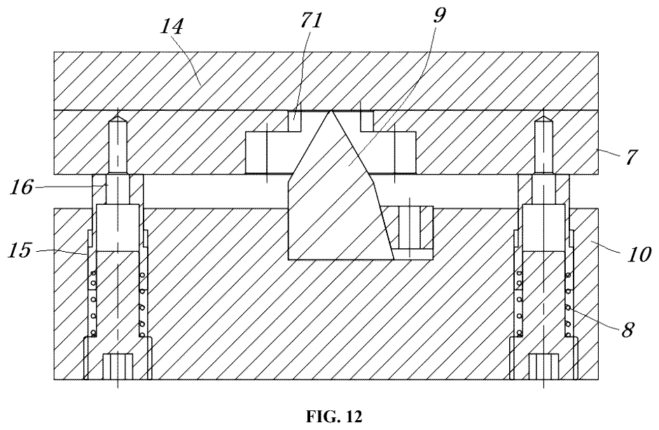

In this embodiment, the upper surface of the telescopic positioning plate 7 is further provided with two positioning blocks 12, as shown in FIG. 9 and FIG. 11. The two positioning blocks 12 are perpendicular to each other, and the two positioning blocks 12 are located on two adjacent sides of the cuboid sample 14. In actual application, the telescopic positioning plate 7 needs to be selected according to the size of the cuboid sample 14, and the telescopic positioning plates 7 with different sizes of cutting-edge exposure holes 71 are selected. The two positioning blocks 12 on the telescopic positioning plate 7 are enclosed to form a sample groove, the prepared cuboid sample 14 should be strictly placed into the sample groove (i.e., two side edges of one right angle of the cuboid sample 14 are respectively abutted against the two positioning blocks 12), thus ensuring that the axis of the cuboid sample 14 coincides with the axis of the lower fixing block 10. After the cuboid sample 14 is placed on the telescopic positioning plate 7, the cuboid sample 14 drives the telescopic positioning plate 7 vertically downwards under the action of self-weight of the cuboid sample 14 until the bottom of the cuboid sample 14 is abutted against the cutting edge of the lower knife 9 as shown in FIG. 12, and the telescopic positioning plate 7 no longer displaces.

Then, the upper fixture is mounted, the upper fixing block 3 is mounted on the guide rods 5, thus ensuring that the upper fixing block 3 can move freely along the guide rods 5 after the upper fixing block 3 is mounted. Prior to test, it should be ensured that the upper knife 4 of the upper fixing block 3 is far away from the sample, or slowly approaches the cuboid sample 14 to preventing the cuboid sample 14 from being damaged before the test starts.

If an axial strain needs to be measured, the axial strain sensor 6 needs to be mounted between the sensor fixing seats 11 of the upper fixing block 3 and of the lower fixing block 10.

Finally, the mounted fixture and the cuboid sample 14 are placed on the indirect tensile tester, the ball head 1 is placed in the spherical groove on the upper portion of the upper fixing block 3, the indirect tensile test can be carried out.

The use process and working principle of the sample fixture 100 for the indirect tensile test of rock mass in this embodiment are similar to or the same as those in Embodiment 1, and thus will not be described in detail here.

Specific examples are used herein for illustration of the principles and embodiments of the present disclosure. The description of the embodiments is merely used to help illustrate the method and its core principles of the present disclosure. In addition, those of ordinary skill in the art can make various modifications in terms of specific embodiments and scope of application in accordance with the teachings of the present disclosure. In conclusion, the content of this description shall not be construed as a limitation to the present disclosure.

What is claimed is:

1. A sample fixture for an indirect tensile test of rock mass, comprising an upper fixture and a lower fixture,
wherein the upper fixture comprises an upper fixing block (3), a ball head (1), and an upper knife (4), a spherical groove adapted for an outer arc surface of the ball head (1) is formed on a top of the upper fixing block (3), and the ball head (1) is embedded into the spherical groove; a bottom of the ball head (1) is in contact and fits with the spherical groove, and a center of a contact surface between the ball head (1) and the spherical groove is located at a central axis of the spherical groove; the upper knife (4) is arranged below the upper fixing block (3), a cutting edge of the upper knife (4) faces downwards, and the cutting edge of the upper knife (4) is aligned with the central axis of the spherical groove; the ball head (1) is configured to be connected to a loading power source to transfer a loading force exerted by the loading power source to the upper fixing block (3) and the upper knife (4) in sequence along the central axis of the spherical groove;
the lower fixture is arranged below the upper fixture, and comprises a lower fixing block (10), a telescopic positioning plate (7), and a lower knife (9); the lower knife (9) is arranged on the lower fixing block (10), a cutting edge of the lower knife (9) faces upwards, and the cutting edge of the lower knife (9) is vertically aligned and parallel with the cutting edge of the upper knife (4); the telescopic positioning plate (7) is located above the lower knife (9) and the lower fixing block (10), and the telescopic positioning plate (7) is connected to the lower fixing block (10) by a telescopic adjusting assembly; the telescopic positioning plate (7) is configured for placing a sample to be tested thereon, and a cutting-edge exposure hole (71), for the cutting edge of the lower knife (9) to make contact with the sample to be tested, is formed in the telescopic positioning plate (7); a central line of the cutting-edge exposure hole (71) in a length direction thereof is vertically aligned and parallel with the cutting edge of the lower knife (9); the telescopic adjusting assembly is configured for driving the telescopic positioning plate (7) to move towards or away from the lower fixing block (10), so as to make the cutting edge of the lower knife (9) in contact with or away from the sample to be tested on the telescopic positioning plate (7).

2. The sample fixture for an indirect tensile test of rock mass according to claim 1, wherein when the sample to be tested is a cylindrical sample (13), the cutting-edge exposure hole (71) also serves as a positioning structure for the cylindrical sample (13), the cylindrical sample (13) is arranged parallel to the length direction of the cutting-edge exposure hole (71), and a lowermost part of the cylindrical sample (13) is embedded into the cutting-edge exposure hole (71) to limit both sides of the cylindrical sample (13) by two long side edges of the cutting-edge exposure hole (71); and when the sample to be tested is a cuboid sample (14), positioning blocks (12) are arranged on an upper surface of the telescopic positioning plate (7), the positioning blocks (12) are distributed on an outer side of the cuboid sample (14) to laterally limit the cuboid sample (14).

3. The sample fixture for an indirect tensile test of rock mass according to claim 2, wherein the positioning blocks (12) arranged on the upper surface of the telescopic positioning plate (7) comprises two positioning blocks (12), and the two positioning blocks (12) are respectively located on two adjacent sides of the cuboid sample (14).

4. The sample fixture for an indirect tensile test of rock mass according to claim 1, wherein the upper fixing block (3), the telescopic positioning plate (7) and the lower fixing block (10) are arranged in parallel, the upper fixing block (3) is connected to the lower fixing block (10) through a vertical guide mechanism, and the vertical guide mechanism is configured to vertically guide the upper fixing block (3) when the upper fixing block (3) moves up and down relative to the lower fixing block (10).

5. The sample fixture for an indirect tensile test of rock mass according to claim 4, wherein the vertical guide mechanism comprises a plurality of guide rods (5) which are parallel to the central axis of the spherical groove, a bottom end of each of the plurality of guide rods (5) is connected to the lower fixing block (10), and a top end of each of the plurality of guide rods (5) movably penetrates through the upper fixing block (3) to be in sliding fit with the upper fixing block (3); and the upper fixing block (3) is connected to the lower fixing block (10) by the plurality of guide rods (5).

6. The sample fixture for an indirect tensile test of rock mass according to claim 5, wherein upper fixing block side bumps (31) are respectively arranged on two sides of the upper fixing block (3), and lower fixing block side bumps (101) are respectively arranged on two sides of the lower fixing block (10); the lower fixing block side bumps (101) are in one-to-one correspondence with the upper fixing block side bumps (31) vertically; each of the plurality of guide rods (5) is connected between a corresponding one of the lower fixing block side bumps (101) and a corresponding one of the upper fixing block side bumps (31), and a bearing (2) is arranged in each of the upper fixing block side bumps (31), and each of the upper fixing block side bumps (31) is in sliding fit with the top end of a corresponding one of the plurality of guide rods (5) by the bearing (2).

7. The sample fixture for an indirect tensile test of rock mass according to claim 1, further comprising an axial strain sensor (6), wherein the axial strain sensor (6) is connected between the upper fixing block (3) and the lower fixing block (10), and is configured to measure a displacement and deformation of the sample to be tested in a vertical direction in a loading process.

8. The sample fixture for an indirect tensile test of rock mass according to claim 7, wherein sensor fixing seats (11) are arranged on sides of both the upper fixing block (3) and the lower fixing block (10), and the sensor fixing seats (11) on the upper fixing block (3) and the lower fixing block (10) are in one-to-one correspondence vertically; and a top end and a bottom end of the axial strain sensor (6) are respectively connected to the sensor fixing seats (11) of the upper fixing block (3) and of the lower fixing block (10).

9. The sample fixture for an indirect tensile test of rock mass according to claim 1, wherein the telescopic adjusting assembly comprises positioning holes (15), springs (8), and positioning shafts (16); the positioning holes (15) are formed in the lower fixing block (10), the springs (8) are embedded into the positioning holes (15), and bottom ends of the springs (8) are coupled or connected to hole bottoms of the positioning holes (15); the positioning shafts (16) are movably inserted into the positioning holes (15), top ends of the positioning shafts (16) are connected to the telescopic positioning plate (7), and bottom ends of the positioning shafts (16) are coupled or connected to top ends of the springs (8), or the bottom ends of the positioning shafts (16) are inserted into the springs (8), limiting bumps are arranged on outer side walls of the positioning shafts (16), and the top ends of the springs (8) are coupled or connected to the limiting bumps; the telescopic positioning plate (7) is able to compress the springs (8) under an action of gravity of the sample to be tested or the loading force exerted by the loading power source to enable the telescopic positioning plate (7) to move towards the lower fixing block (10); and after the sample to be tested is taken down, the telescopic positioning plate (7) is capable of being far away from the lower fixing block (10) under a reset rebound effect of the springs (8).

10. The sample fixture for an indirect tensile test of rock mass according to claim 1, wherein at least one of the cutting edge of the upper knife (4) and the cutting edge of the lower knife (9) is a round-headed cutting edge.

11. The sample fixture for an indirect tensile test of rock mass according to claim 2, wherein the upper fixing block (3), the telescopic positioning plate (7) and the lower fixing block (10) are arranged in parallel, the upper fixing block (3) is connected to the lower fixing block (10) through a vertical guide mechanism, and the vertical guide mechanism is configured to vertically guide the upper fixing block (3) when the upper fixing block (3) moves up and down relative to the lower fixing block (10).

12. The sample fixture for an indirect tensile test of rock mass according to claim 3, wherein the upper fixing block (3), the telescopic positioning plate (7) and the lower fixing block (10) are arranged in parallel, the upper fixing block (3) is connected to the lower fixing block (10) through a vertical guide mechanism, and the vertical guide mechanism is configured to vertically guide the upper fixing block (3) when the upper fixing block (3) moves up and down relative to the lower fixing block (10).

13. The sample fixture for an indirect tensile test of rock mass according to claim 2, further comprising an axial strain sensor (6), wherein the axial strain sensor (6) is connected between the upper fixing block (3) and the lower fixing block (10), and is configured to measure a displacement and deformation of the sample to be tested in a vertical direction in a loading process.

14. The sample fixture for an indirect tensile test of rock mass according to claim 3, further comprising an axial strain sensor (6), wherein the axial strain sensor (6) is connected between the upper fixing block (3) and the lower fixing block (10), and is configured to measure a displacement and deformation of the sample to be tested in a vertical direction in a loading process.

15. The sample fixture for an indirect tensile test of rock mass according to claim 2, wherein the telescopic adjusting assembly comprises positioning holes (15), springs (8), and positioning shafts (16); the positioning holes (15) are formed in the lower fixing block (10), the springs (8) are embedded into the positioning holes (15), and bottom ends of the springs (8) are coupled or connected to hole bottoms of the positioning holes (15); the positioning shafts (16) are movably inserted into the positioning holes (15), top ends of the positioning shafts (16) are connected to the telescopic positioning plate (7), and bottom ends of the positioning shafts (16) are coupled or connected to top ends of the springs (8), or the bottom ends of the positioning shafts (16) are inserted into the springs (8), limiting bumps are arranged on outer side walls of the positioning shafts (16), and the top ends of the springs (8) are coupled or connected to the limiting bumps; the telescopic positioning plate (7) is able to compress the springs (8) under an action of gravity of the sample to be tested or the loading force exerted by the loading power source to enable the telescopic positioning plate (7) to move towards the lower fixing block (10); and after the sample to be tested is taken down, the telescopic positioning plate (7) is capable of being far away from the lower fixing block (10) under a reset rebound effect of the springs (8).

16. The sample fixture for an indirect tensile test of rock mass according to claim 3, wherein the telescopic adjusting assembly comprises positioning holes (15), springs (8), and positioning shafts (16); the positioning holes (15) are formed in the lower fixing block (10), the springs (8) are embedded into the positioning holes (15), and bottom ends of the springs (8) are coupled or connected to hole bottoms of the positioning holes (15); the positioning shafts (16) are movably inserted into the positioning holes (15), top ends of the positioning shafts (16) are connected to the telescopic positioning plate (7), and bottom ends of the positioning shafts (16) are coupled or connected to top ends of the springs (8), or the bottom ends of the positioning shafts (16) are inserted into the springs (8), limiting bumps are arranged on outer side walls of the positioning shafts (16), and the top ends of the springs (8) are coupled or connected to the limiting bumps; the telescopic positioning plate (7) is able to compress the springs (8) under an action of gravity of the sample to be tested or the loading force exerted by the loading power source to enable the telescopic positioning plate (7) to move towards the lower fixing block (10); and after the sample to be tested is taken down, the telescopic positioning plate (7) is capable of being far away from the lower fixing block (10) under a reset rebound effect of the springs (8).

17. The sample fixture for an indirect tensile test of rock mass according to claim 2, wherein at least one of the cutting edge of the upper knife (4) and the cutting edge of the lower knife (9) is a round-headed cutting edge.

18. The sample fixture for an indirect tensile test of rock mass according to claim 3, wherein at least one of the cutting edge of the upper knife (4) and the cutting edge of the lower knife (9) is a round-headed cutting edge.
